(19) United States
(12) Patent Application Publication
(10) Pub. No.: US 2005/0178394 A1
(43) Pub. Date: Aug. 18, 2005

(54) Method for keratophakia surgery

(75) Inventor: Stephen G. Slade, Houston, TX (US)

Correspondence Address: FULBRIGHT & JAWORSKI, LLP, 1301 MCKINNEY, SUITE 5100, HOUSTON, TX, 77010-3095, US

(73) Assignee: Intralens Vision, Inc.

(21) Appl. No.: 10/924,152

(22) Filed: Aug. 23, 2004

Related U.S. Application Data
(60) Provisional application No. 60/496,797, filed on Aug. 21, 2003.

(51) Int. Cl. A61B 18/20; A61B 19/00

(52) U.S. Cl. 128/898; 606/4

(57) ABSTRACT

The invention relates to surgical methods for performing eye surgery to correct focusing deficiencies of the cornea. More particularly, the present invention relates to the surgical procedure known as keratophakia, and related surgical methods for performing lamellar keratotomies and refractive surgery. The method is designed to create a flap of epithelium, or stroma and epithelial flap as is done now with Keratophakia but with shapes to aid surgical centration, surgical stability, long-term stability of the lens and maintaining the corrective power. This will enable keratophakia and its advantages of removability and potential reversibility to improve its outcomes to patients.

This application claims priority to U.S. Provisional application No. 60/496,797 filed on Aug. 21, 2003.

### TECHNICAL FIELD

The present invention relates to surgical methods for performing eye surgery to correct irregularities of the cornea. More particularly, the present invention relates to the procedure of Keratophakia with synthetic or natural tissue, and related surgical methods for using a femtosecond laser or other keratome to create a specific flap to improve centration and stability of the lenticle.

### BACKGROUND OF THE INVENTION

The eye works on a principle very similar to that of a camera. The iris or colored portion of the eye about the pupil, functions like a shutter to regulate the amount of light admitted to the interior of the eye. The cornea and natural lens focus the rays of light on the retina. The retina then transmits the image of the object viewed to the brain via the optic nerve. Normally, these light rays will be focused exactly on the retina, which permits the distant object to be seen distinctly and clearly. Deviations from the normal shape of the corneal surface, however, produce errors of refraction in the visual process so that the eye becomes unable to focus the image of the distant object on the retina. Hyperopia or “farsightedness” is an error of refraction in which the light rays from a distant object are brought to focus at a point behind the retina, as indicated by the solid lines. Myopia or “nearsightedness” is an error of refraction in which the light rays from a distant object are brought to focus in front of the retina, as indicated by the solid lines, such that when the rays reach the retina, they become divergent, forming a circle of diffusion and consequently, a blurred image.

In recent years, as refractive surgery has developed, a number of surgical techniques have become available to surgically treat near sightedness, farsightedness and astigmatism. Of these surgical techniques, the first technique developed by Barraquer was the procedure of Keratophakia for myopia, hyperopia and astigmatism. Referring to FIGS. 2A and 2B, Keratophakia has the advantage of being an “additive” technique, where no tissue is removed from the patient. Adding substance creates the refractive effect. Thus, the material or substance may be removed or replaced if the desired effect is not achieved or if time or other changes makes the removal or exchange desirable. LASIK, a more common technique, does remove tissue and as such permanent alters the eye and is not reversible or removable.

Referring to FIG. 3-5, Keratophakia surgery consists of cutting a flap of cornea stroma and epithelium, lifting the flap and placing a lens of some material, organic or synthetic on the exposed bed. The flap is then repositioned and seals itself down. As shown in FIG. 4, a microkeratome is typically used to fashion the flap. The microkeratome is generally a blade carrying device which functions like a carpenter's plane or surgical dermatome, that may be manually pushed or mechanically driven a cutting path across a suction ring simultaneous with the motorized movement of the cutting element, which movement is transverse to the direction of the cutting path.

More recently a laser has been developed to make the flap. This is a femtosecond laser and is capable of making multiple types of flap. See FIG. 6 showing a flap being made with a femtosecond laser. The depth, diameter, hinge position and other variables may be set with the software of the laser enabling an infinite variety of flap/bed shapes.

Jose Ignacio Barraquer at his clinic in Bogota, Columbia began fifty years ago to develop the concept of lamellar refractive corneal surgery. He conceptualized that by removing corneal stroma tissue or adding tissue that the tear film-anterior cornea interface would be affected and alter the refractive power of the eye. He reported his first results in 1949. The surgical term for the techniques that Dr. Barraquer developed was keratophakia, which is derived from the Greek roots keras (horn-like—cornea) and phakia (lens).

Dr. Barraquer's initial technique consisted of performing a freehand lamellar dissection with a Paufique Knife or corneal dissector to create a corneal lamellar disc. He then shaped a refractive lens from human stroma from a donor using a cryolathe.

With Dr. Barraquer's groundbreaking work and subsequent research into sculpting a corneal disc with a cryolathe he laid the basis for modern myopic keratomileusis, hyperopic keratomileusis, keratophakia and LASIK.

Several drawbacks, however, were inherent to Dr. Barraquer's initial techniques, which included the complex nature of the procedure and instrumentation, low margin for error, suitable lens material and steep surgeon learning curve. Additionally, since the keratectomies were done by free hand, the resection depended on a steady rate of passage, adequate suction and good cent ration. If a good keratectomy was not achieved interface scarring, an irregularly thin corneal disc and ultimately irregular astigmatism could be experienced.

The critical steps for achieving the best results in keratophakia are the controlled pass of the microkeratome and a suitable synthetic material for the lenticle. Luis Ruiz in the late 1980's developed a foot operated automated geared microkeratome. This microkeratome provided a more consistent cut due to controlled speed and the keratectomy displayed a very smooth surface. Subsequently new and better keratomes such as the Hansatome and the femtosecond laser have added precision and safety to lamellar surgery.

Researchers have used many materials in an attempt to find a suitable substitute for human tissue. Human tissue is limited in availability and must be shaped while frozen. For example, a synthetic material such as that discussed in U.S. Pat. No. 6,102,946 may be porous to nutrients, and able to be produced in a variety of shapes and powers in great precision.

With modern keratomes and the femtosecond laser, flaps can be created that are smooth and precise reducing the chance for irregular astigmatism. Modern materials may reduce rejection or inflammation of the cornea and provide nutrients transmission. Keratophakia lenticles may be made for the correction of hyperopia, myopia and astigmatism in any combination.

Keratophakia has several potential advantages over other subtractive techniques such as LASIK. LASIK surgery consists of cutting a flap of cornea stroma and epithelium, lifting the flap and reshaping the exposed bed with an excimer laser. The flap is then repositioned and seals itself down. Since the patient's cornea is not altered in a refractive nature in Keratophakia, the operation is more “reversible.” Once the lens is placed, if a change is needed the flap can be lifted and a new lens placed after the old one is removed. This adds safety to the procedure. In addition, LASIK and Keratophakia can be combined for a variety of refractive errors.

New work on keratophakia has shown that the problems of irregular astigmatism and nutrient transmission are largely solved. Now, the attention is the critical centration of the lenticle and the need to place the lenticle in such a way that it does not move after placement. The lens must be centered precisely and then its centration must be maintained during the replacement of the flap. Further, the lens must remain in its desired site after the surgery which can be difficult.

Further, using Keratophakia to address astigmatism means that the lens must not only remain centered but must not torque or rotate.

A surgical method is proposed to fashion a flap with a femtosecond laser quickly and reproducibly with a minimum of manipulation to the cornea, lifting the flap, implanting a lens, and the replacing the flap over the lens. As one skilled in the art would understand after reading the disclosure herein, the flap could be made in an infinite variety of shapes to promote centration and stability of the lens.

### SUMMARY OF THE INVENTION

The present invention is designed to satisfy a need in lamellar surgery and is directed towards a new and improved method for making a keratophakia flap with a microkeratome or femtosecond laser system. In addition, the present invention is designed to cut into the cornea to create a hinged flap of corneal tissue, for example under the Bowman's membrane or into the cornea stroma. An appropriately selected lens is implanted on the exposed corneal tissue, and then the flap is placed over the lens.

In one embodiment, the positioning means comprise a design for the flap to position a lens designed to address hyperopia or farsightedness. In keratophakia for hyperopia, a circular, convex lens or one that is thicker in the middle than the edges is placed in the bed. The present invention describes a design of flap and bed so that a “Recess” or a “Platform” or a “Rail” or a “Gutter” or “Oval” or “Post” or “Tab” configuration is created to allow easy, rapid and precise centration during the surgery, stability during the replacement of the flap and stability of the lens after the surgery.

In one embodiment of the invention there is a method of implanting a corneal implant. The method includes separating a portion of the outer surface of a cornea thereby forming a corneal flap and a corneal bed. The has an anterior surface and a posterior surface. The corneal bed has a shaped anterior surface. A lens, such as a corneal implant, is implanted or placed on the corneal bed. Typically, the lens has an anterior surface and a posterior surface. Many types of lenses would be known to one skilled in the art that would be appropriate for use with the inventive method. The portion of the cornea that was separated is replaced.

There are several important aspects to this method. In one aspect of the invention the corneal bed is shaped in a configuration to maintain the lens in position during surgery.

In one aspect of the invention the corneal bed is shaped to aid in centration of the lens during the surgery and/or after surgery.

In one aspect of the invention, the corneal bed is shaped to match the shape of the posterior surface of the lens.

In one aspect of the invention implanting of the lens and replacing the corneal flap corrects for hyperopia, myopia, or astigmatism.

In another aspect, the corneal bed and the corneal flap are shaped or configured so that they form a mirror image of one another. The following shapes, not intended to be an exhaustive list, are useful to aid in centration of the lens. Such shapes includes, the anteriot surface of the corneal bed having a recess configuration, platform configuration, gutter configuration, rail configuration, oval configuration, tab configuration, or a post configuration.

In one aspect of the invention, a femtosecond laser is utilized to create a geometrically specific flap to aid placement of the lens. Currently centration over the visual axis is difficult with Keratophakia. The patient is not able to fixate once the corneal flap is made reliably. Thus different light reflexes must be relied upon. A mechanical keratome may not make a perfectly centered flap. Using a femtosecond laser to make the corneal flap allows the flap to be centered directly over the pupil, a preferred method for refractive centration. Since this flap is centered, the shape may also be centered. So that the lens may be placed in the centered shaped and it will be in the desired position.

Another aspect of the invention is the creation of a shape that will aid in stabilizing the lens during the replacement of the flap. In keratophakia surgery, replacing the flap can dislodge the lens as well as irrigating the interface. The lens in the current method will be protected from this.

Another aspect of the invention is the long-term stability afforded the lens. It has been noted in modern Keratophakia that the lens can change its position some time after surgery. This may be due to excess fluid or other factors. However, the creation of the flap and corneal bed as described herein, provides long-term stability during surgery and after surgery.

Another aspect of the invention is the ability to position the lens to reduce or prevent torquing or rotating. By the use of an oval shape, a rectangle, other shapes or tabs could keep the lens in position.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

A better understanding of the invention can be obtained from the detailed description of exemplary embodiments set forth below, when considered in conjunction with the appended drawings, in which:

FIG. 1 is a schematic illustration of the cornea;

FIGS. 2A-B are illustrations of the additive effect of keratophakia;

FIG. 3 is an illustration of keratophakia;

FIG. 4 is an illustration of flap being cut;

FIG. 5 is an illustration of a flap being lifted after cut;

FIG. 6 is an illustration of the femtosecond laser flap;

FIGS. 7, 7A-B are illustrations of a corneal flap and bed in a recess configuration;

FIGS. 8, 8A-B are illustrations of a corneal flap and bed in a platform configuration;

FIGS. 9, 9A-B are illustrations of a corneal flap and bed in a rail configuration;

FIGS. 10, 10A-B are illustrations of a corneal flap and bed in a gutter configuration;

FIG. 11 is an illustration of the geometry of astigmatism;

FIGS. 12, 12A are illustrations of a corneal flap and bed in an oval configuration;

FIGS. 13, 13A are illustrations of a corneal flap and bed in a tab configuration; and

FIG. 14, 14A are illustrations of a corneal flap and bed in a post configuration.

### BRIEF DESCRIPTION OF THE DRAWINGS

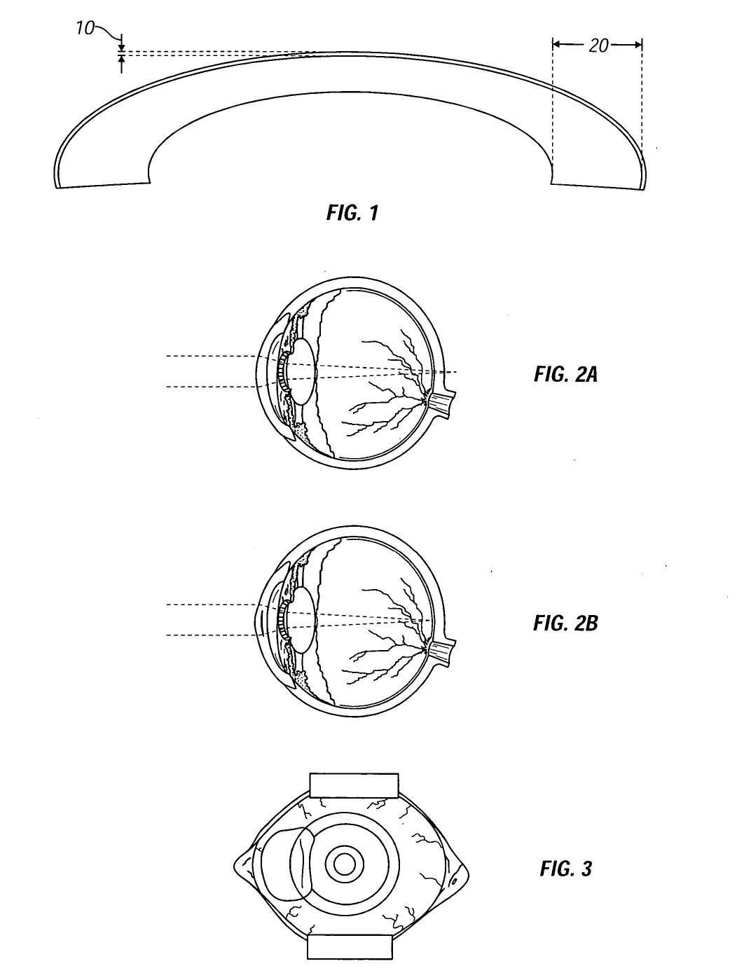

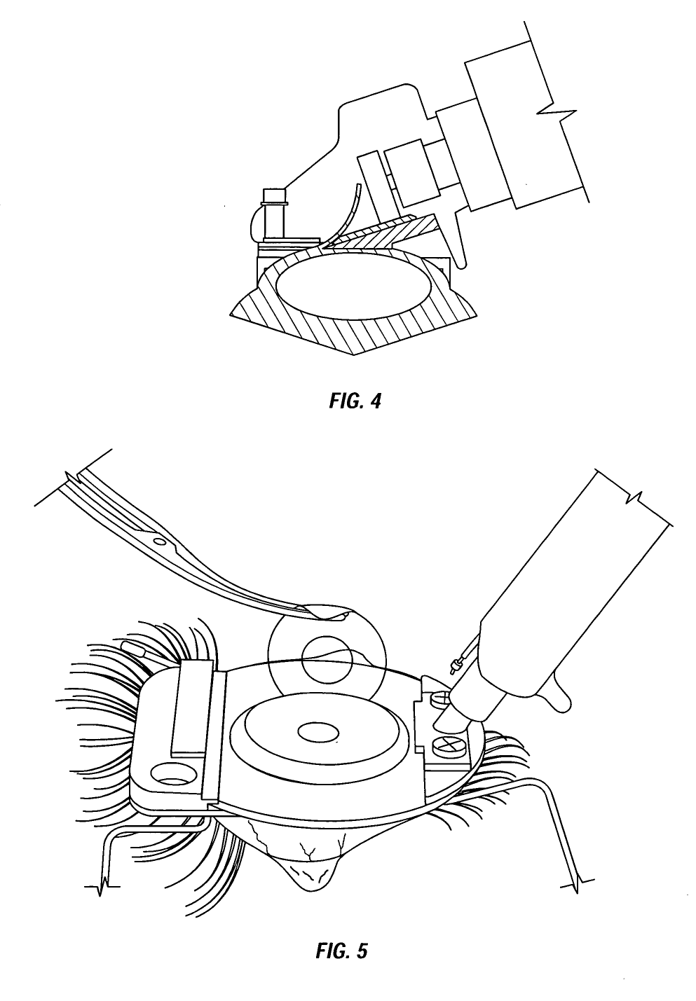

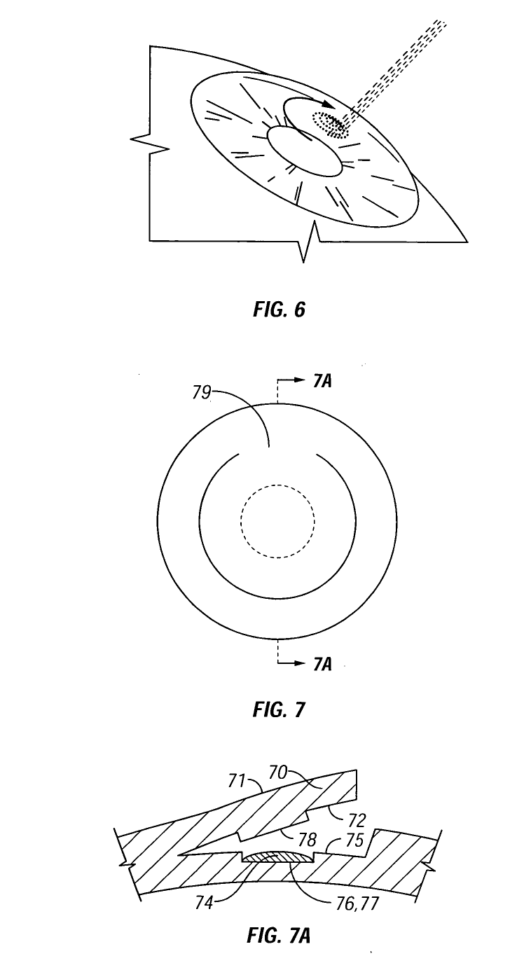

FIG. 1 is a schematic illustration of the cornea. The cornea or clear window of the eye, and the lens, which is located behind the pupil, serve to focus the light rays from an object being viewed onto the retina at the back of the eye. The cornea is composed of five layers; first the epithelium that is five cells thick and is usually around 60 microns thick 10-10. A thin membrane called Bowman's membrane underlies the epithelium. The mass of the cornea is called the stroma, which is about 480 microns thick 20. The fourth layer is another, stronger but very thin membrane called Descemet's. The final layer is the endothelium, which is only one cell thick. Bowman's, Descemet's and the endothelium do not contribute significantly to the total cornea thickness. The total thickness of the cornea averages around 540 microns. Once the cornea and lens focus the rays of light on the retina, the retina then transmits the image of the object viewed to the brain via the optic nerve. Normally, these light rays will be focused exactly on the retina, which permits the distant object to be seen distinctly and clearly. Deviations from the normal shape of the corneal surface, however, produce errors of refraction in the visual process so that the eye becomes unable to focus the image of the distant object on the retina. Hyperopia or “farsightedness” is an error of refraction in which the light rays from a distant object are brought to focus at a point behind the retina, as indicated by the solid lines. Myopia or “nearsightedness” is an error of refraction in which the light rays from a distant object are brought to focus in front of the retina, as indicated by the solid lines, such that when the rays reach the retina, they become divergent, forming a circle of diffusion and consequently, a blurred image.

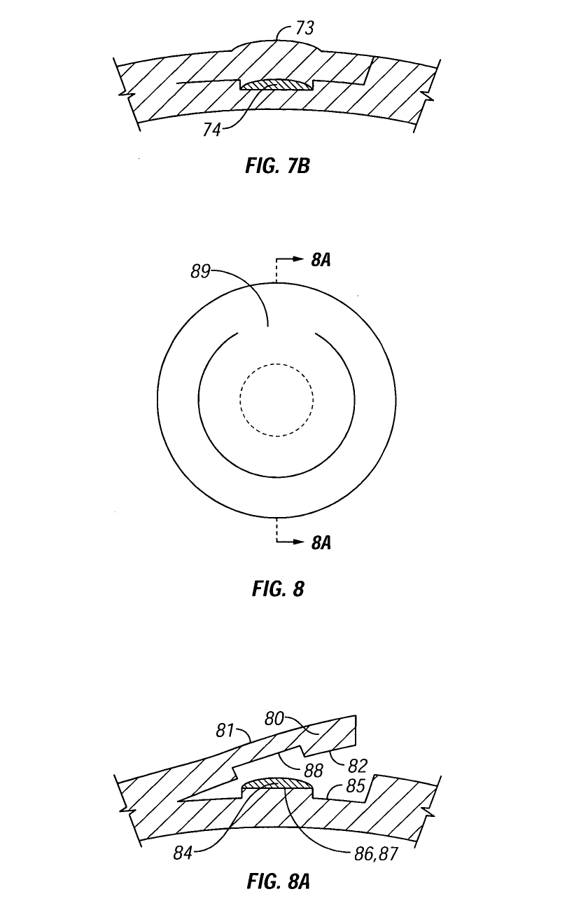

Referring now to FIG. 7, the recess structure is a shaped cut into the bed of the cornea during the creation of the flap. The corneal flap 70 has an anterior surface 71 and a posterior surface 72. The exposed surface of the cornea is referred to as the corneal bed 75. The exposed corneal bed has an anterior surface 76. The recess is created in any position to address astigmatism, in any depth or diameter desired. The lens 79 is placed in the recess 77 and the flap is repositioned. Since a mirror image of the recess is created on the underside of the flap, the refractive effect would be preserved. In an example of the particular embodiment shown, the corneal flap and the corneal bed are a mirror image of one another. The corneal flap is shaped such that the flap has a protrusion 78 that fits with a well of the corneal bed. A lens or corneal implant 74 is placed in the recess and the corneal flap laid back down. The protrusion of the corneal flap lays in contact with the implant. This has the effect of steepening the cornea 73. The top-view shown shows the corneal flap having a hinge 79.

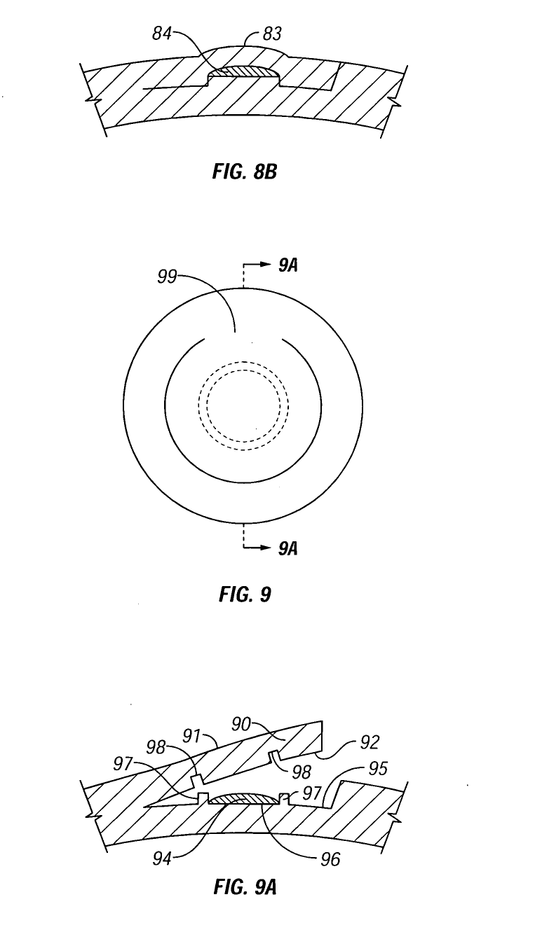

Referring now to FIG. 8, the platform structure is a raised shaped created on the corneal bed in any position, shape or dimension. The corneal flap 80 has an anterior surface 81 and a posterior surface 82. The exposed surface of the cornea is referred to as the corneal bed. 85 The exposed corneal bed has an anterior surface 86. A lens 84 is placed on the platform 87 and the flap is repositioned. Since a mirror image of the platform is created on the underside of the flap, the refractive effect would be preserved. In an example of the particular embodiment shown, the corneal flap and the corneal bed are a mirror image of one another. The corneal flap is shaped such that the flap has a recess 88 that fits with a platform of the corneal bed. A lens or corneal implant is placed on the platform and the corneal flap laid back down. The recess of the corneal flap lays in contact with the implant. This has the effect of steepening the cornea 83. The top-view shown shows the corneal flap having a hinge 89.

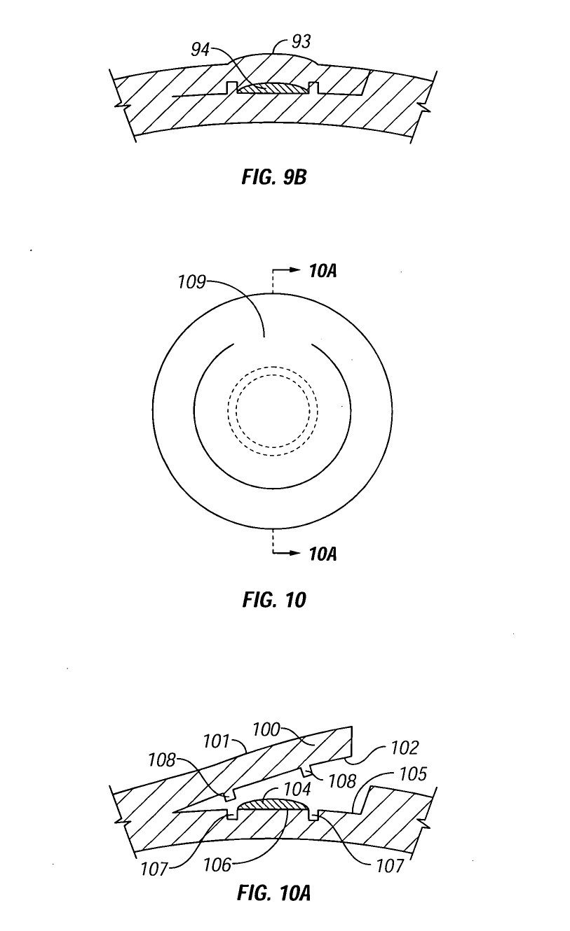

Referring now to FIG. 9, the rail structure is a raised circular band or fence created on the bed in any position, shape or dimension. The corneal flap 90 has an anterior surface 91 and a posterior surface 92. The exposed surface of the cornea is referred to as the corneal bed. 95 The exposed corneal bed has an anterior surface 96. The lens is placed within the rail 97 and the flap is repositioned. Since a mirror image of the rail is created on the underside of the flap, the refractive effect would be preserved. In an example of the particular embodiment shown, the corneal flap and the corneal bed are a mirror image of one another. The corneal flap is shaped such that the flap has a gutter or channel 98 that fits the raised circular band or fence of the corneal bed. A lens or corneal implant 94 is placed within the fence or rail and the corneal flap laid back down. The surface of the corneal flap between the gutter or channel lays in contact with the implant. This has the effect of steepening the cornea 93. The top-view shown shows the corneal flap having a hinge 99.

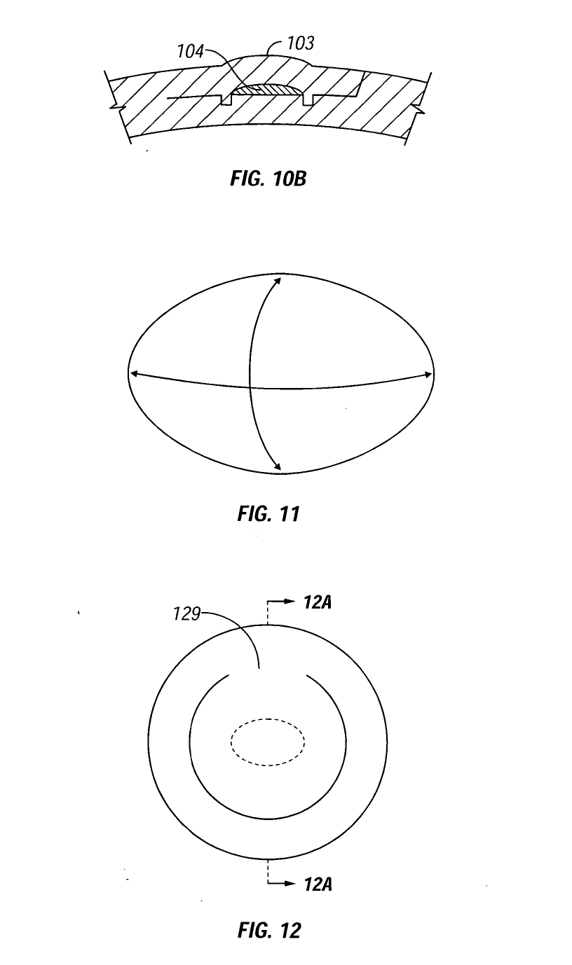

Referring now to FIG. 10, the gutter structure is a lowered channel created on the bed in any position, shape or dimension. The corneal flap 100 has an anterior surface 101 and a posterior surface 102. The exposed surface of the cornea is referred to as the corneal bed. 105 The exposed corneal bed has an anterior surface 106. The lens 104 is placed centered within a gutter or channel 107 and the flap is repositioned. Since a mirror image of the gutter is created on the posterior surface of the flap, the refractive effect would be preserved. In an example of the particular embodiment shown, the corneal flap and the corneal bed are a mirror image of one another. The corneal flap is shaped such that the flap has a fence or rail 108 that fits with a gutter or channel of the corneal bed. A lens or corneal implant is placed on a surface between the gutter or channel of the corneal bed and the corneal flap laid back down. The surface of the corneal flap between the fence or rail lays in contact with the implant. This has the effect of steepening the cornea 103. The top-view shown shows the corneal flap having a hinge 109.

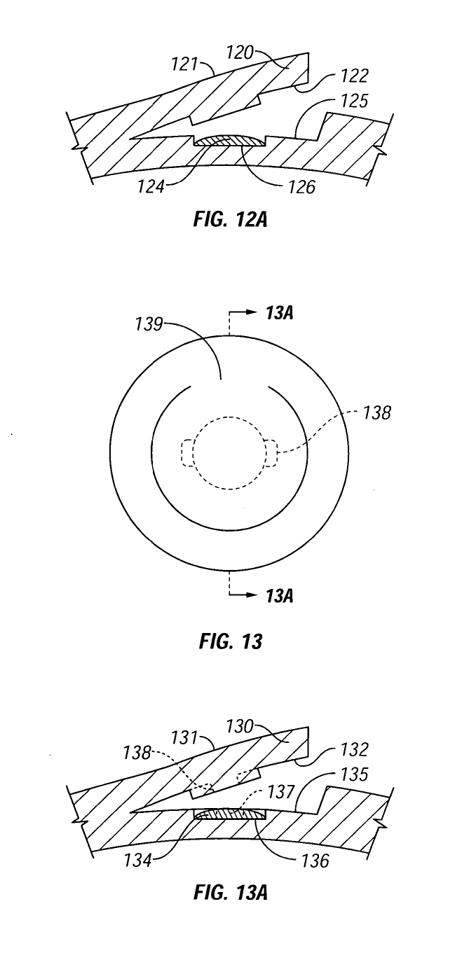

Keratophakia may also be used to correct astigmatism. FIG. 11 is an illustration of the geometry of astigmatism. To correct for astigmatism, a lens is required that is steeper or flatter in one axis than the other. This lens 124 may be oval shaped. Referring to FIGS. 12 and 12A, in such a lens a combination of the above shapes may be made in an oval fashion to add centration, maintain centration during the replacement of the flap, aid postoperative stability but also keep the astigmatic lenticle from torquing or rotating. The corneal flap 120 has an anterior surface 121 and a posterior surface 122. The exposed surface of the cornea is referred to as the corneal bed. 125 The exposed corneal bed has an anterior surface 126. If a specific lens was created with higher order aberration correction then these techniques may be used to center it too.

Referring to FIGS. 13 and 13A, in addition to prevent rotation of the lens, a further shape tab may be created in any dimension, size or location. In one example, but not to limit the tab shape 138, the tab may have the shapes as shown. The corneal flap 130 has an anterior surface 131 and a posterior surface 132. The exposed surface of the cornea is referred to as the corneal bed. 135 The exposed corneal bed has an anterior surface 136.

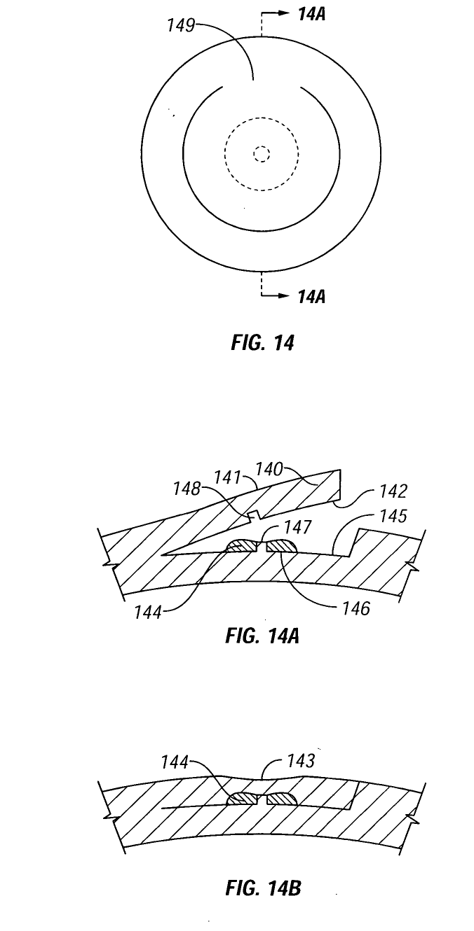

Keratophakia may also be used to correct myopia. In myopia the lens is thinner or even not existent in the center area compared to the periphery. Referring to FIGS. 14, 14A, a lens 144 with a central opening can be stabilized by a post 147. The corneal flap 140 has an anterior surface 141 and a posterior surface 142. The exposed surface of the cornea is referred to as the corneal bed. 145 The exposed corneal bed has an anterior surface 146. The corneal bed has a post extending from the anterior surface of the bed. A lens that has an opening in placed over the post. The post has a corresponding slot 148 in the corneal flap. The top-view shown shows the corneal flap having a hinge 149. A lens with a thinner portion in the center will have a flattening effect 143.

Included in the invention is the ability to fashion any combination in any dimension of any flap or of the above basic shapes. Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

Moreover, the embodiments described are further intended to explain the best modes for practicing the invention, and to enable others skilled in the art to utilize the invention in such, or other, embodiments and with various modifications required by the particular applications or uses of the present invention. It is intended that the appending claims be construed to included alternative embodiments to the extent that it is permitted by the prior art.

1. A method of implanting a corneal implant, comprising the steps of:
separating a portion of the outer surface of a cornea thereby forming a corneal flap and a corneal bed, said corneal flap having an anterior surface and a posterior surface, said corneal bed having a shaped anterior surface;
implanting a lens on said corneal bed, said lens having an anterior surface and a posterior surface; and
replacing the portion of the cornea that was separated.

2. The method of claim 1, further comprising:
shaping the corneal bed in a configuration to maintain said lens in position.

3. The method of claim 1, further comprising:
shaping the corneal bed to aid in centration of said lens during the surgery.

4. The method of claim 1, further comprising:
shaping the corneal bed to aid in stabilizing the lens during the replacement of the corneal flap.

5. The method of claim 1, wherein the shaped anterior surface is a recess configuration.

6. The method of claim 1, wherein the shaped anterior surface is a platform configuration.

7. The method of claim 1, wherein the shaped anterior surface is a gutter configuration.

8. The method of claim 1, wherein the shaped anterior surface is a rail configuration.

9. The method of claim 1, wherein the shaped anterior surface is an oval configuration.

10. The method of claim 1, wherein the shaped anterior surface is a tab configuration.

11. The method of claim 1, wherein the shaped anterior surface is a post configuration.

12. The method of claim 11, wherein the lens has a central opening.

13. The method of any one of claims 1-10 wherein the lens has a middle that is thicker than an edge of the lens.

14. The method of any one of claims 1-10 wherein the separating step includes utilizing a keratome to form the corneal flap.

15. The method of any one of claims 1-10 wherein the separating step includes utilizing a femtosecond laser system to form the corneal flap.

16. The method of any one of claims 1-10, wherein the separating step includes:
shaping the shaped corneal bed to match the shape of the posterior surface of the lens.

17. The method of any one of claims 1-10, wherein implantation of said lens and the corneal flap corrects for hyperopia.

18. The method of any one of claims 1-10, wherein implantation of said lens and the corneal flap corrects for myopia.

19. The method of any one of claims 1-10, wherein implantation said lens and the corneal flap corrects for astigmatism.

20. The method of any one of claims 1-10, wherein said lens has two axes, and the lens is steeper in one axis, and flatter in the other axis.
